(19) United States
(12) Patent Application Publication
(10) Pub. No.: US 2009/0306341 A1
(43) Pub. Date: Dec. 10, 2009

(54) LC/MS Blends Containing Ionizing Additives

(76) Inventor: Joachim Emmert, Mannheim (DE)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/989,246

(22) PCT Filed: Jul. 27, 2006
(86) PCT No.: PCT/DE2006/001304

§ 371 Date: Feb. 10, 2009

(30) Foreign Application Priority Data
Jul. 29, 2005 (DE) 20 2005 011 957.1
May 31, 2006 (DE) 20 2006 008 638.2

(51) Int. Cl. C07K 1/16; G01N 30/72

(52) U.S. Cl. 530/344; 252/408.1

(57) ABSTRACT

The invention relates to the preparation of a salt in acetonitrile, characterised in that the acid component of the salt is an organic acid which boils at less than 300° C. under normal pressure, the base component of the salt is a base which boils at less than 300° C. under normal pressure, an organic acid which boils at less than 300° C. under normal pressure is added in the quantity of up to 1 vol. %, in relation to the volume of acetonitrile, and the water content, that can be determined by Karl Fischer titration, is below 5%.

LC/MS, i.e. the coupling of liquid chromatography with mass spectrometry, has been the method of choice for several problems in the areas of pharmaceutical, clinical and forensic chemistry as well as environmental analysis and in the area of food monitoring since the 1990s. Especially in the areas of biotechnology and proteomics, LC/MS has developed into one of the most important analytical techniques, in particular for the separation and structural determination of proteins.

Electron spray ionization (ESI) is the most widely used ionization technique in LC/MS of proteins and peptides, whereby the addition of ionizing aids or ionizing additives has been proved successful. These ionizing additives facilitate the transition of the analytes into charged particles (ions) in the gas phase as is described in LC-GC Europe, 17(12), pages 646 to 649, 2004. One of the most widely used additives in normal HPLC is phosphoric acid and its sodium or potassium salts as it allows for an excellent adjustment of pH values due to its three buffering stages. The salts of phosphoric acid are, however, not volatile, thus do not support ionization and form a precipitate at the shield of the ionization chamber, where they must sometimes be removed using great effort.

Organic acids such as formic acid, acetic acid and propionic acid have been shown to be suitable ionizing additives. However, in this case, the separation occurs in the acidic range with almost exclusively positive ionization under formation of [M+H]⁺ ions.

As a mild alternative, the use of ammonium salts of formic acid and acetic acid, in particular of ammonium acetate has proved to be advantageous; thus pH values can be controlled, which allow for separation and ionization under mild conditions as well as for positive and negative ionization. Moreover, it is characterized by its good volatility, i.e. no or hardly any residues remain in the ionization chamber under typical ESI ionization conditions of approximately 350° C. at atmospheric pressure.

Another difficulty is the presence of alkali ions which markedly affect the absolute sensitivity even at low concentrations of 5 to 6 ppm (cf. also LC-GC Europe, 17(12), pages 646 to 649, 2004). These alkali ions can be added to the solvent by starting materials that are not sufficiently pure or, in case water or methanol is used, also by elution from the glass walls. By use of acetonitrile, the glass is subjected to such elution to a markedly lower extent; amongst other reasons, it is therefore preferably used.

Thus, it would be advantageous to provide a mixture of an ionizing additive and a solvent, a so-called solvent blend, which does not form any residues in the ionization chamber and is as low as possible in alkali ions.

However, blends comprising salt-like ionization additives such as ammonium acetate in acetonitrile are at present only prepared by addition of 5 to 10% of water as ammonium salts of lower organic acids such as formic acid, acetic acid or propionic acid are not soluble in pure acetonitrile and water is necessary as a co-solvent. Further, it is necessary to prepare the mixtures shortly before use as the ammonium acetate comprising solvent mixture is characterized by a poor shelf life and poor pH stability below a water content of 5% due to ammonia leaking from the solution. This high water content also results in the previously mentioned undesired phenomenon of elution.

Furthermore, it is impossible to obtain a gradient value of 100% acetonitrile with a water-containing acetonitrile blend. For instance, with an ammonium acetate-containing blend in acetonitrile prepared by addition of 10% water, a gradient value of only 0 to 90% can be realized.

Thus, the object of the invention was to provide salt-like ionizing additive containing blends in acetonitrile which are low in water and sodium, are characterized by a good shelf life and pH stability and which form hardly any or no residues in the ionization chamber of a mass spectrometer.

This object is solved by adding an excess of an organic acid to the salt-like ionizing additive in such a way that the base can be kept in a protonated state. Moreover, the added organic acid serves as a co-solvent in such a way that the ionizing additive is soluble even in the absence of water.

Acid suitable as acid components of the salt-like ionizing additive comprise volatile organic acids. Sufficiently volatile acids possess a boiling point of below 300° C. at atmospheric pressure, more preferably below 250° C. and most preferably below 200° C. Preferred embodiments are formic acid, acidic acid and propionic acid.

Bases suitable as base components of the salt-like ionizing additive comprise volatile weak bases. Sufficiently volatile bases possess a boiling point below 300° C. at atmospheric pressure, more preferably below 250° C. and most preferably below 200° C. Preferred embodiments are ammonia, methylamine, ethylamine, n-propylamine, isopropylamine, dimethylamine, diethylamine, trimethylamine and triethylamine.

Organic acids serving as a co-solvent comprise volatile organic acids. Sufficiently volatile acids possess a boiling point below 300° C. at atmospheric pressure, more preferably below 250° C. and most preferably below 200° C. Preferred embodiments are formic acid, acetic acid and propionic acid.

The ionizing additive can be used in any amount until the saturation point of the additive in the blend is reached. Preferably, the ionizing additive is used in an amount of 0.001 to 3% (w/v), more preferably in an amount of 0.01 to 2% (w/v) and most preferably in an amount of 0.1 to 1% (w/v).

The organic acid serving as a co-solvent can be used in any amount. The organic acid is preferably used in an amount of up to 1 vol.-% based on the volume of acetonitrile, more preferably 8 to 32 equivalents based on the molar amount of the ionization additive and most preferably 10 to 16 equivalents.

The water content of the LC/MS mixtures according to the present invention is preferably between 0 to 5%, more preferably between 0.1% and 4%, even more preferably between 0.5% and 3% and most preferably between 1% and 2% and can be determined by conventional methods, for example Karl Fischer titration.

The purity of the used substances is preferably high enough to allow for the use of the mixtures according to the present invention in chromatography (HPLC) and LC/MS. In particular, it is preferred that the substances used are so pure that organic impurities, as for instance softening agents, are largely absent, i.e. the content of organic impurities is lower than 10 ppm, more preferably lower than 6 ppm and most preferably lower than 1 ppm.

It is also preferred that the substances used are so pure that the content of alkali and earth alkaline ions is lower than 5 ppm per type of ion, more preferably lower than 2 ppm per type of ion and even more preferably lower than 1 ppm per type of ion and most preferably lower than 0.2 ppm per type of ion. Further, it is preferred that the overall alkali content is below 0.25 ppm, more preferably below 0.2 ppm and most preferably below 0.15 ppm.

The process of making in particular the blend sequence is not limited. In particular, it is possible to add the ionizing additive as a salt or to form the salt in situ by separate addition of the acid and base during its making. The product obtained can optionally be filtrated under sterile conditions.

### DESCRIPTION OF THE DRAWINGS

FIG. 1 shows the pH gradient of the mixtures according to the present invention and comparative mixtures. The pH gradients A, B, C and D comprise the following components:

A: water; 0.1% ammonium acetate/pure acetonitrile
B: water; 0.1% ammonium acetate/methanol; 0.1% ammonium acetate
C: water; 0.1% ammonium acetate/acetonitrile; 0.1% ammonium acetate
D: pure water/acetonitrile; 0.1% ammonium acetate

FIG. 2 shows the overall MS ion chromatogram of the separation of bradykinines (BK). Peaks 1 to 5 represent:
1: BK 1-6
2: Lys-Ala3-BK
3: BK
4: Des-Arg1-BK
5: impurity

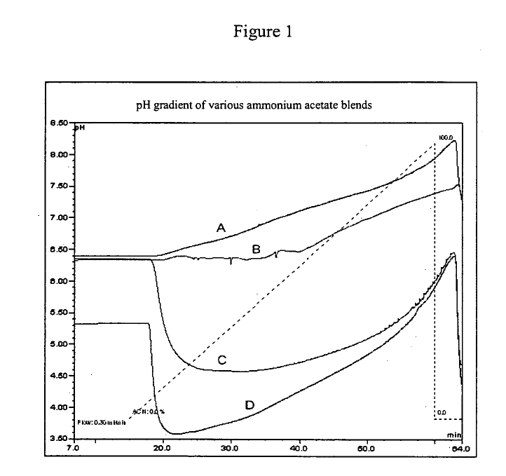

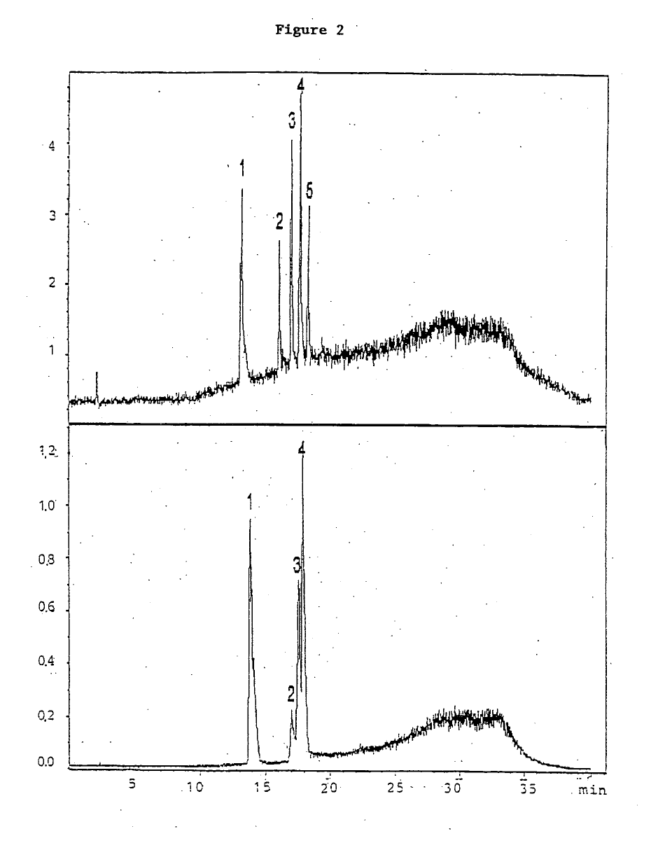

PH gradients of pH 3.5 to pH 6.5 can be realized with the help of LC/MS mixtures according to the present invention, which allow for positive and negative ionization during chromatographic separation. As can be seen from FIG. 1, a constantly increasing gradient of pH 3.5 to pH 6.5 can be realized.

The solvent blends for LC/MS according to the present invention allow for chromatographic separation of very similar peptides (bradykinines (BK)) under slightly acidic conditions (FIG. 2, top chromatogram, condition C). This separation is much less efficient under conventional neutral to weakly alkaline conditions (FIG. 2, chromatogram at the bottom, condition A).

When using the ESI ionization chamber with the mixtures according to the present invention, markedly decreased deposits of residues were found.

Subsequently, examples of making the LC/MS blends according to the present invention will be given. The numbers in parentheses behind the adducts refer to product numbers of the Fluka-Riedel catalogue published in 2005.

### PREPARATION EXAMPLE 1

An LC/MS blend comprising a 0.1% (w/v) ammonium acetate as ionizing additive was prepared as follows.

A volume part of an aqueous 10 percent by weight ammonium acetate solution (#32301, #34877) is added to a glass apparatus and with stirring, a volume part of acetic acid (#33209) is added. Subsequently, 98 volume parts of acetonitrile (#34697) are added in order to obtain a blend for LC/MS with an ionizing additive concentration of 0.1% (w/v). The mixture is filtered under sterile conditions through a 0.2 μm filter. The clear colorless solution obtained possesses a water content below 2.0%, as determined by Karl Fischer titration, and a sodium content lower than 2 ppm and potassium, magnesium and calcium contents lower than 0.5 ppm, respectively.

### PREPARATION EXAMPLE 2

An LC/MS blend comprising a 0.05% (w/v) ammonium acetate as an ionizing additive was prepared as follows.

100 liters of acetonitrile (#34967) and a volume part of acetic acid (#33209) are added to a glass apparatus and cooled. Subsequently, 0.65 moles of ammonium are added with stirring in order to obtain a blend for LC/MS with an ionizing additive concentration of 0.05% (w/v). The clear colorless solution obtained possesses a water content below 0.01%, as determined by Karl Fischer titration, and a sodium content of less than 1 ppm and potassium, magnesium and calcium contents lower than 0.5 ppm, respectively.

### PREPARATION EXAMPLES 3 TO 8

LC/MS blends comprising 0.1% (w/v) of ammonium acetate as an ionizing additive were prepared as in example 1, apart from amendments to the compositions as shown in table 1 and optional addition of water.

TABLE 1

| | | 10% ammonium | Water | Acetic acid |
|---|---|---|---|---|
| | Acetonitrile | acetate solution | (volume | (volume |
| Example | (volume parts) | (volume parts) | parts) | parts) |
| 3 | 95 | 1 | 4 | 0 |
| 4 | 96 | 1 | 3 | 0 |
| 5 | 96 | 1 | 2.5 | 0.5 |
| 6 | 96 | 1 | 2 | 1 |
| 7 | 97 | 1 | 1 | 1 |
| 8 | 98 | 1 | 0 | 1 |

The alkali content of the solutions obtained was determined with the help of ICP-OES and ICP-MS. The results shown in table 2 were obtained:

TABLE 2

| | Na [ppb] | | K [ppb] | |
|---|---|---|---|---|
| Example | ICP-OES | ICP-MS | ICP-OES | ICP-MS |
| 3 | 161 | 200 | 46 | <10 |
| 4 | 158 | 180 | 43 | <10 |
| 5 | 141 | 150 | 96 | <10 |
| 6 | 174 | 180 | 43 | <10 |
| 7 | 100 | 165 | 25 | <10 |
| 8 | 127 | 140 | 46 | <10 |

From table 2 it can be seen that the overall alkali content of the examples according to the present invention is lower than 0.25 ppm and preferably lower than 0.2 ppm.

The solutions obtained according to examples 3 to 8 were used for the chromatographic separation of very similar peptides (bradykinines (BK)). The following results were obtained.

TABLE 3

| | Retention times peptides [min] | | | |
|---|---|---|---|---|
| Example | BK Fr. 1-6 | Lys-Ala3-BK | Bradykinin | Des-Arg1-BK |
| 3 | 13.7 | 17.7 | 17.9 | 18.1 |
| 4 | 13.6 | 17.7 | 17.8 | 17.9 |
| 5 | 13.2 | 16.3 | 17.1 | 17.7 |
| 6 | 13.2 | 16.3 | 17.1 | 17.7 |
| 7 | 13.1 | 16.2 | 17.0 | 17.6 |
| 8 | 13.0 | 16.1 | 16.9 | 17.6 |

From table 3 it can be seen that the HPLC separation occurs more efficiently in the presence of low water and alkali ion content.

1. A solution of a salt in acetonitrile, wherein the acid component of the salt is an organic acid boiling below 300° C. under atmospheric pressure, and the base component of the salt is a base boiling below 300° C. under atmospheric pressure, and an organic acid boiling below 300° C. under atmospheric pressure is added in an amount of up to 1 vol.-% based on the volume of acetonitrile, wherein the solution comprises less than 5% water, which can be determined by Karl Fischer titration.

2. The solution according to claim 1, wherein the acid component of the salt is selected from formic acid, acetic acid, propionic acid and trifluoroacetic acid, the base component of the salt is selected from ammonia, methylamine, ethylamine, n-propylamine, isopropylamine, dimethylamine, diethylamine, trimethylamine and triethylamine and the organic acid boiling below 300° C. under atmospheric pressure is selected from formic acid, acetic acid and propionic acid.

3. The solution according to claim 1, wherein the organic acid boiling below 300° C. at atmospheric pressure is added in an amount of 8 to 32 equivalents based on the molar amount of the salt.

4. The solution according to claim 1, wherein the water content is below 2%.

5. The solution according to claim 1, wherein the salt is selected from ammonium formate, ammonium acetate and ammonium propionate.

6. The solution according to claim 1, wherein the acetonitrile possesses HPLC purity or LC/MS purity.

7. The solution according to claim 1, wherein the solution was filtered under sterile conditions.

8. The solution according to claim 1, further comprising an amount of alkali and earth alkaline ions below 2 ppm per type of ion.

9. The solution according to claim 1, wherein the solution comprises less than 10 ppm of organic impurities.

10. The solution according to claim 1, wherein the solution comprises the salt in an amount of 0.001 to 3% (w/v).

11. A solution of a salt obtained by the process of mixing the acid component of the salt selected from organic acids boiling below 300° C. under atmospheric pressure, the base component of the salt selected from bases boiling below 300° C. under atmospheric pressure and an organic acid boiling below 300° C. under atmospheric pressure and used in an amount of up to 1 vol.-% based on the volume of acetonitrile, wherein the obtained mixture comprises less than 5% water, which can be determined by Karl Fischer titration.

12. The solution according to claim 11, wherein the acid component of the salt and the base component of the salt are added together as one salt.

13. The solution according to claim 11, wherein the acid component of the salt is selected from formic acid, acetic acid, propionic acid and trifluoroacetic acid, the base component of the salt is selected from ammonia, methylamine, ethylamine, n-propylamine, isopropylamine, dimethylamine, diethylamine, trimethylamine and triethylamine and the organic acid boiling below 300° C. under atmospheric pressure is selected from formic acid, acetic acid and propionic acid.

14. The solution according to claim 11, wherein the solution was filtered under sterile conditions.

15. A method for chromatographic separation, characterized by the use of the solution according to claim 1.

16. The method for the chromatographic separation according to claim 15, wherein peptides are chromatographed.

17. Use of the solution according to claim 1 in chromatography.

18. Use according to claim 17, wherein peptides are chromatographed.
